(19) United States
(12) Patent Application Publication
(10) Pub. No.: US 2017/0319750 A1
(43) Pub. Date: Nov. 9, 2017

(54) COMPOSITE MATRICES DESIGNED FOR ENHANCED BONE REPAIR

(71) Applicants: BIOVENTUS, LLC, Durham, NC (US); CollPlant Ltd., Ness Ziona (IL)

(72) Inventors: Orly Grinberg, Modi'in (IL); Nadav Orr, Mazkeret Batia (IL); Howard Seeherman, Cambridge, MA (US); Oded Shoseyov, Shoham (IL); Eric Vanderploeg, Stoneham, MA (US); Christopher Wilson, Auburndale, MA (US); John Wozney, Hudson, MA (US)

(21) Appl. No.: 15/589,005

(22) Filed: May 8, 2017

Related U.S. Application Data
(60) Provisional application No. 62/333,571, filed on May 9, 2016.

(51) Int. Cl. A61L 27/48; A61L 27/12; A61L 27/18; A61L 27/24; B29C 39/02; A61L 27/54; A61L 27/56; A61L 27/58; B29C 39/00; A61L 27/34; A61K 38/18; B29K 105/16; B29K 509/02; A61F 2/28; B29L 31/00

(52) U.S. Cl. CPC A61L 27/48; A61K 38/1875; A61L 27/12; A61L 27/18; A61L 27/24; B29C 39/025; A61L 27/54; A61L 27/56; A61L 27/58; B29C 39/003; A61L 27/34; A61L 2300/414; A61L 2420/02; A61L 2430/02; B29K 2089/00; B29K 2105/16; B29K 2509/02; A61F 2002/2835; B29L 2031/7532; B29K 2995/006

(57) ABSTRACT

Osteoconductive synthetic bone grafts are provided in which porous ceramic granules are embedded in a biocompatible matrix material. The grafts, which may also include one or more of a coating, a reinforcing bio-absorbable mesh, and an osteoinductive protein or peptide, are generally porous and may incorporate fenestrations.

### FIELD OF THE INVENTION

This application relates to medical devices and biologic therapies, and more particularly to bone cements, bone putties and ceramic-binder composites.

### RELATED APPLICATIONS

This application claims the benefit of priority under 35 U.S.C. §119(e) to U.S. Provisional Application No. 62/333,571, filed May 9, 2016, which is hereby incorporated by reference in its entirety.

### BACKGROUND

Bone grafts are used in roughly two million orthopedic procedures each year, and general take one of three forms. Autografts, which typically consist of bone harvested from one site in a patient to be grafted to another site in the same patient, are the benchmark for bone grafting materials, inasmuch as these materials are simultaneously osteoconductive (serving as a scaffold for new bone growth), osteoinductive (promoting the development of osteoblasts) and osteogenic (containing osteoblasts which form new bone). However, limitations on the supply of autografts have necessitated the use of cadaver-derived allografts. These materials are less ideal than autografts, however, as allografts may trigger host-graft immune responses or may transmit infectious or prion diseases, and are often sterilized or treated to remove cells, eliminating their osteogenicity.

Given the shortcomings of human-derived bone graft materials, there has been a long-standing need in the field for synthetic bone graft materials. Synthetic grafts typically comprise calcium ceramics and/or cements delivered in the form of solid or granular implants, a paste or a putty. These materials are osteoconductive, but not osteoinductive or osteogenic. To improve their efficacy, synthetic calcium-containing materials have been loaded with osteoinductive materials, particularly bone morphogenetic proteins (BMPs), such as BMP-2, BMP-7, or other growth factors such as fibroblast growth factor (FGF), insulin-like growth factor (IGF), platelet-derived growth factor (PDGF), and/or transforming growth factor beta (TGF-β). However, significant technical challenges have prevented the efficient incorporation of osteoinductive materials into synthetic bone graft substitutes which, in turn, has limited the development of high-quality osteoinductive synthetic bone graft materials.

One such challenge has been the development of a graft matrix which delivers an osteoinductive material over time, rather than in a single short burst release, and which has appropriate physical characteristics to support new bone growth. The generation of a material with appropriate physical characteristics involves, among other things, balancing the requirement that such materials be rigid enough to bear loads that will be applied to the graft during and after implantation with the requirements that they remain porous enough to allow for cell and tissue infiltration and degrade or dissolve at a rate which permits replacement of the graft by new bone, and the separate requirement that they elute the osteoinductive material in a temporal and spatial manner that is appropriate for bone generation. It is only the combination of the above design criteria that will result in an optimal graft matrix for promoting new bone formation and ultimate healing. For example, BMP-eluting synthetic bone grafts currently available commercially do not meet these requirements, and a need exists for a bone graft material which is optimized for the delivery of osteoinductive materials such as BMPs.

### SUMMARY OF THE INVENTION

The present invention addresses these needs by providing synthetic bone graft materials capable of effective delivery of osteoinductive proteins in combination with optimal physical characteristics, as well as methods of making and using the same. In one aspect, the present invention includes a biocompatible scaffold configured for the in vivo delivery of osteoinductive proteins. In certain embodiments, the scaffold comprises an rhCollagen matrix having a plurality of calcium ceramic granules therein, and a bioresorbable polymer mesh. In certain embodiments, the scaffold further comprises an rhCollagen coating.

In another aspect, the present invention includes a method of making the biocompatible scaffolds of the present invention.

In another aspect, the present invention includes a kit that comprises the biocompatible scaffolds of the present invention.

In yet another aspect, the present invention includes a method of treating a patient by contacting a bony tissue of the patient with the biocompatible scaffolds of the present invention.

### BRIEF DESCRIPTION OF THE DRAWINGS

Certain embodiments of the present invention are illustrated by the accompanying figures. It will be understood that the figures are not necessarily to scale and that details not necessary for an understanding of the invention or that render other details difficult to perceive may be omitted. It will be understood that the invention is not necessarily limited to the particular embodiments illustrated herein.

FIG. 1 shows the in vitro retention profile (% of initial) for BMP-2 delivered from calcium deficient hydroxyapatite (CDHA) porous high Specific Surface Area (SSA) granules, CDHA non-porous high SSA granules, macroporous calcium phosphate cement (CaP), 60:40 HA/tricaldium phosphate (TCP) porous granules, 15:85 HA/TCP porous granules as a function of time in days, and an absorbable collagen sponge (ACS). BMP was loaded onto the carriers in BMP buffer solution for 1 hour. The BMP-loaded granules were then incubated in a solution containing 20% bovine serum to mimic exposure to serum proteins in vivo. High specific surface area CDHA granules with and without porosity and CaP cements had superior BMP in vitro retention compared to ACS and low SSA granules either alone or contained within a collagen sponge.

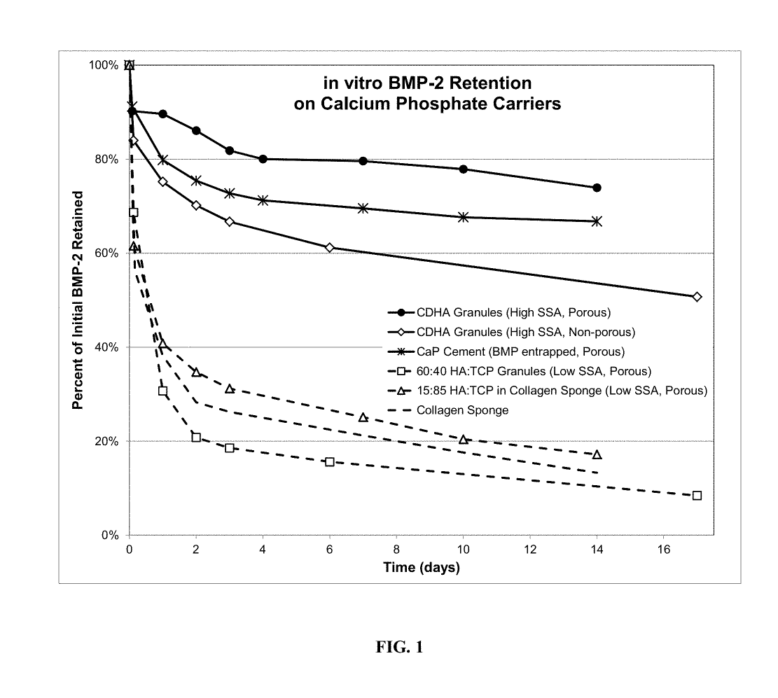

FIG. 2 shows the in vivo BMP retention profile of a scaffold of an embodiment of the present invention (labelled as “Biocompatible Composite Matrix”) in a rodent muscle pouch model compared to a commercially available collagen sponge (labelled as “Collagen Sponge”). As illustrated by the figure, the scaffold, which included an rhCollagen matrix containing ceramic calcium phosphate granules and a polymer mesh in accordance with embodiments of the present invention, has the ability to retain BMP at the site of implantation for extended time periods compared to a matrix consisting only of collagen.

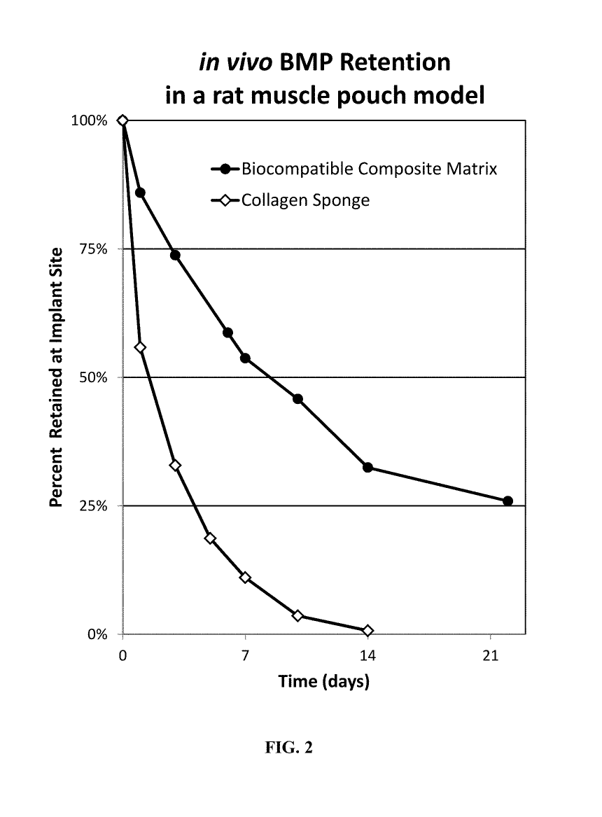

FIGS. 3A-D show the inherent macroporosity of the composite constructs of embodiments of the present invention with clearly identifiable pores between 100-300 microns in size. A photograph (FIG. 3A) and micro-computed tomography (micro-CT) image (FIG. 3B) demonstrate the space between granules that is supported by the biocompatible matrix. Higher magnification micro-CT (FIG. 3C) and SEM images (FIG. 3D) reveal the inherent macropores throughout the construct seen between the rhCollagen fibers and the ceramic calcium phosphate granules.

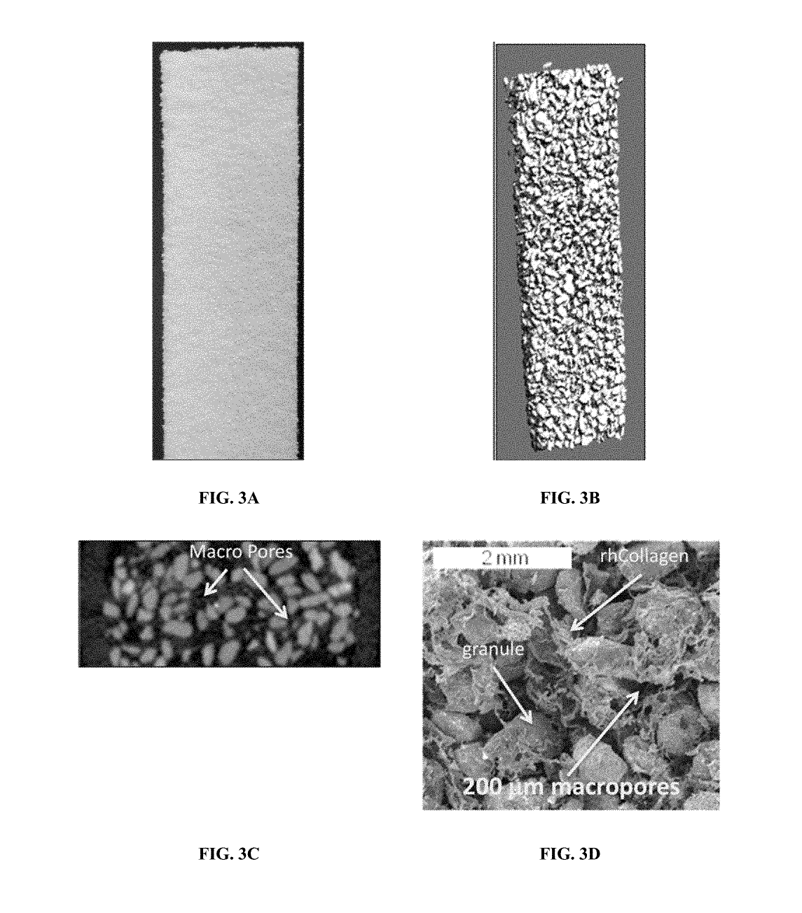

FIG. 4 includes a histological section that demonstrates that the biocompatible and macroporous nature of the composite constructs of the present invention enables new bone formation to occur uniformly throughout the implant in a rodent muscle pouch model. Bone is evident between and adjacent to the ceramic calcium phosphate granules within the implant.

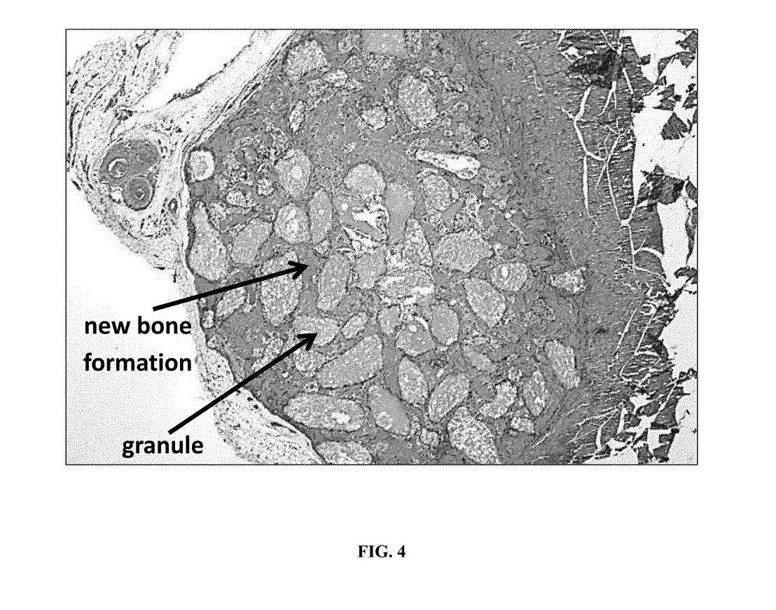

FIGS. 5A-C show an exemplary composite construct of an embodiment of the present invention with a fenestration pattern introduced during the molding process. Photographic (FIG. 5A) and Micro-CT images (FIGS. B and C) reveal the macropores between individual calcium phosphate granules and the open fenestrations (holes) that span the width of the construct.

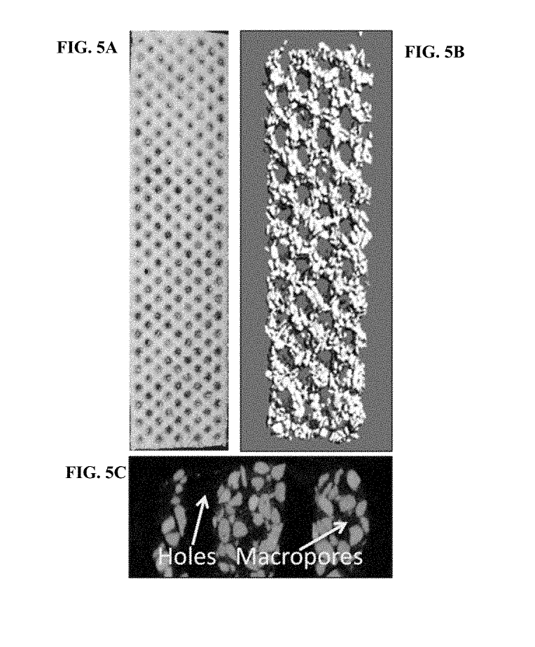

FIGS. 6A and B show photographs of a construct of an embodiment of the present invention with an applied surface coating and demonstrate the ability of the surface coating to reduce ceramic granule shedding during handling.

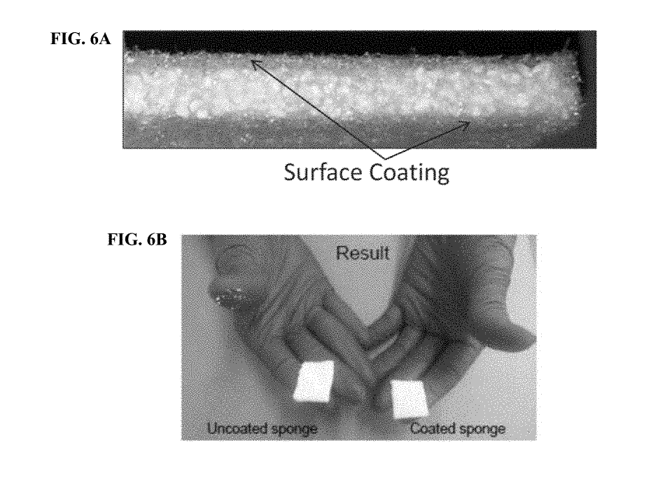

FIGS. 7A and B show photographs of an exemplary reinforcing element (in FIG. 7B), in accordance with an embodiment of the present invention, embedded with a construct (as shown in FIG. 7A) to provide improved mechanical integrity.

FIGS. 8A-C show molds that are used to manufacture scaffolds of the present invention.

FIGS. 9A-B show in-life CT images and explant micro-computed tomography (XT) (9A) and histology images (9B) from a non-human primate treated with the composite constructs of the present invention in a posterolateral fusion model at 24 weeks post-surgery.

### DETAILED DESCRIPTION

### Osteoinductive Compositions

Synthetic bone grafts (also referred to interchangeably herein as “implants,” “constructs” and “scaffolds”) of the present invention generally include three components: an osteoconductive material such as a calcium ceramic or other solid mineral body, an osteoinductive material such as a bone morphogenetic protein, and a biocompatible matrix such as a collagen sponge. As used herein, osteoconductive materials refer to any material which facilitates the ingrowth or ongrowth of osteoblastic cells including osteoblasts, pre-osteoblasts, osteoprogenitor cells, mesenchymal stem cells and other cells which are capable of differentiating into or otherwise promoting the development of cells that synthesize and/or maintain skeletal tissue. In preferred embodiments of the present invention, the osteoconductive material includes granules comprising an osteoconductive calcium phosphate ceramic that is adapted to provide sustained release of an osteoinductive substance that is loaded into or onto the granules. In some cases, the granules include interconnected, complex porous structures. Exemplary granules, which the inventors have found exhibit BMP binding and elution characteristics that are optimized for use in constructs, systems and methods of the present invention, are described in U.S. Provisional Patent Application No. 62/097,363 by Vanderploeg et al., the entire disclosure of which is incorporated by reference herein for all purposes. These granules are associated (or “loaded”) with osteoinductive materials such as BMPs using methods known in the art. For example, the osteoinductive material, such as a BMP, is applied to the granules, or a matrix containing the granules, as a liquid solution and the osteoinductive material absorbs to the surfaces (both interior and exterior surfaces) of the granules.

Osteoinductive materials generally include peptide and non-peptide growth factors that stimulate the generation of osteoblasts from populations of pre-cursor cells. In some embodiments, the osteoinductive material is a member of the transforming growth factor beta (TGF-B) superfamily such as BMP-2, BMP-3, BMP-4, BMP-5, BMP-6, BMP-7, BMP-9, or a designer BMP such as the BMP-GER or BMP-GER-NR chimeric BMPs described in U.S. Pre-grant application publication no. US 20120046227 A1 by Berasi et al. entitled “Designer Osteoinductive proteins,” the entire disclosure of which is hereby incorporated by reference for all purposes. In other embodiments, the osteoinductive material is a fibroblast growth factor, insulin-like growth factor, platelet-derived growth factor, a small molecule, a nucleotide, a lipid, or a combination of one or more of the factors listed herein.

A third component of implants according to the present invention is the biocompatible matrix, which can be any suitable biocompatible material (a) that, when used in concert with the granules, exhibits sufficient rigidity and/or column strength to withstand the loads placed upon it when implanted, (b) which does not cause excessive inflammation (i.e. inflammation sufficient to inhibit or prevent the formation of new bone or the healing of a broken bone), inhibit the proliferation of osteoblasts, or otherwise interfere with the activity of the granules and/or the osteoinductive material, and (c) has sufficient cohesion over an appropriate interval to permit the deposition of new bone within a defined area. In preferred embodiments, the biocompatible matrix includes fibrillar collagen e.g. a type I, II, III, V, X collagen, a biologically active portion therefrom, or a mixture of one or more such collagens. The collagen(s) are preferably recombinant human collagen (rhCollagen) derived from plant sources, e.g., rhCollagen expressed in a plant and isolated therefrom. Such collagen(s) and their sources and methods of manufacture are described in U.S. Pat. No. 8,455,717; WO 2014/147622; Stein H. et al., “Production of bioactive, post-translationally modified, heterotrimeric, human recombinant type-I collagen in transgenic tobacco,” Biomacromolecules (Impact Factor: 5.75), September 2009; 10(9):2640-45; Shilo S. et al., “Cutaneous wound healing after treatment with plant-derived human recombinant Collagen flowable gel,” Tissue Eng Part A. 2013 July; 19(13-14): 1519-26; Shoseyov O. et al., “Human recombinant type I collagen produced in plants,” Tissue Engineering: Part A Volume 19, Numbers 13 and 14, 2013: 1527-33; and Willard J. J. et al., “Plant-derived human collagen scaffolds for skin tissue engineering,” Tissue Engineering: Part A Volume 19, Numbers 13 and 14, 2013: 1507-18.

In alternative embodiments, the biocompatible matrix includes one or more of the following: hyaluronic acid (HA), and functionalized or modified versions thereof, gelatin (animal or recombinant human), fibrin, chitosan, alginate, agarose, self-assembling peptides, whole blood, platelet-rich plasma, bone marrow aspirate, polyethylene glycol (PEG) and derivatives thereof, functionalized or otherwise cross-linkable synthetic biocompatible polymers including poly(lactide-co-glycolide), poly(caprolactone), poly(lactic acid), poly(glycolic acid), poloxamers and other thermosensitive or reverse-thermosensitive polymers known in the art, and copolymers or admixtures of any one or more of the foregoing.

Where a collagen biocompatible matrix is used, it will be appreciated that the cohesion and column strength of the resulting construct will be determined, at least in part, by the level of cross-linking of the collagen. In preferred embodiments, the collagen(s) used will be cross-linked, preferably by chemical means including, without limitation glutaraldehyde- or carbodiimide-based cross-linking reactions, Dehydrothermal (DHT) treatment or naturally occurring cross-linkers such as genipin. A preferred crosslinking agent is 1-[3-(dimethylamino)propyl]-3-ethylcarbodimide (EDC).

### Technical Considerations for Implant Design

Implants of the invention, which include the osteoinductive materials, granules and biocompatible matrices as described above, generally have characteristics which are tailored to the facilitation of bone growth and healing and which are not exhibited by currently available synthetic bone grafting materials. The relevant characteristics of implants according to the present invention include at least (a) kinetics of release of osteoinductive materials that are appropriate for the application, (b) residence time appropriate to facilitate, but not interfere with new bone formation, (c) macroporosity that permits the infiltration of cells and tissues, including new vascular tissue that accompanies the formation of new bone, and (d) sufficient rigidity and/or compression resistance to withstand loads applied to the implant.

### Retention of Osteoinductive Molecules

Without wishing to be bound by any theory, it is thought that BMPs induce bone formation primarily by stimulating differentiation of osteoblast progenitors either resident at the site of repair in the bone envelope or in the surrounding soft tissue envelope. Physiological bone repairs are stimulated by the release of picogram/femtogram amounts of BMPs stored in the mineral phase of bone and from newly synthesized BMPs secreted by bone progenitor cells at the site of the repair. These two sources of BMP maintain BMP concentrations at the site of repair at physiological levels for the appropriate amount of time to induce a successful bone repair.

In view of this physiology, exogenous BMPs are, ideally, delivered in constructs which elute BMP in therapeutic amounts and over therapeutic intervals that mimic the physiological BMP response. It should be noted, however, that the administration of much larger pharmacological BMP concentrations is generally required to achieve physiological concentrations of BMPs at the cellular level and to maintain the physiological concentrations for the appropriate amount of time. This is due to a combination of factors that are not totally understood. Without wishing to be bound by any theory, one factor driving the need for super-physiological BMP concentrations in these constructs may be the inability of exogenous BMP to mimic the efficiency of physiological local release of endogenous BMPs from bone and newly formed endogenous BMPs from cells. In addition, rhBMPs are generally insoluble at physiological pH, so (again, not wishing to be bound by any theory) much of the exogenously delivered BMP may not be physiologically available.

The amount of exogenous rhBMP required to stimulate bone repair appears to be species dependent. Empirical data suggests that lower concentrations of exogenous rhBMPs are required to stimulate bone formation in small animals such as rodents and rabbits compared to larger animals including dogs, sheep and goats. Nonhuman primates and humans appear to require the highest concentrations of exogenous rhBMPs to stimulate bone repair. For example, the FDA approved concentration of rhBMP-2 delivered in an absorbable collagen sponge (ACS) for bone repair in dogs is 0.2 mg/mL compared to 1.5 mg/mL in humans. Again, the factors contributing to this difference in required exogenous rhBMP concentration are not fully understood, but those of skill in the art will understand that inter-species differences must be considered in evaluating findings in animal models for applicability to human patients.

Similarly, the interval over which BMPs must be delivered to tissues varies among species: BMP residence time for repairs in rodents and rabbits can be as short as several days due to their rapid intrinsic rate of bone formation, while nonhuman primates and human patients generally requires several weeks BMP residence time. While not wishing to be bound by any theory, the longer interval observed in primates and humans appears to be related to the amount of time for the healing process to transition from an initial catabolic inflammatory phase caused by the surgery or trauma to an anabolic phase involving the migration and differentiation of osteoblast progenitors and associated new blood vessel units to support the fusion/repair process. Short BMP residence time optimal for rodents may not maintain physiological BMPs levels for a sufficient amount of time to stimulate bone repair in animals with slower bone formation rates. Conversely, BMP may not be released in sufficient amounts from a carrier with a longer retention profile to stimulate bone formation in animals with rapid intrinsic bone formation rates.

As one example, the residence time of BMPs delivered locally in buffer solution to a repair site is extremely short, and even when relatively large amounts of BMP are delivered in solution, an adequate bone response is only stimulated in rodent models. For applications in non-human primates and human patients, an extended-release carrier is preferably used to localize BMP to sites of treatment for a period of weeks.

One strategy for providing extended local BMP release is to utilize carriers that mimic the binding of BMP to endogenous extracellular matrix. As one example, collagenous carriers exhibit longer BMP residence times than BMP solutions, due (without being bound to any theory) to the intrinsic binding properties of BMP to extracellular matrix components including endogenous collagen. Ceramic carriers including calcium phosphate matrices (CPM) more closely mimic physiologic release of BMP from bone with very long residence times. The release of BMP from ceramic carriers may require the same osteoclastic resorption observed in release of BMP from bone. Based on this unique property, implants comprising ceramic components embedded within composite carriers, as are used in the present invention, may be superior vehicles for BMP delivery compared to other naturally occurring and synthetic biomaterials.

### Structural Considerations

In order to provide temporally and spatially optimal delivery of BMPs, carriers according to the various embodiments of the present invention are preferably macroporous such that they allow penetration of new blood vessels and bone forming cells into the repair site to generate a uniform full thickness repair. Carriers that are not macroporous can result in repairs that have mechanically inferior shells of bone on their surface that do not fully penetrate into the repair. For instance, the ACS used to deliver BMP-2 in INFUSE® has a void volume in excess of 90%. However, the average pore size of that ACS is relatively small, limiting infiltration to individual cells. BMP can also freely diffuse out of the sponge and once BMP is released, the small pore size limits the penetration of blood vessel units into the carrier required to initiate bone formation. As a result, bone formation in response to treatment with BMP-2/ACS generally occurs in the highly vascular granulation tissue outside the resorbing collagen sponge rather than inside the sponge. Rapid mineralization of newly forming bone at the periphery of the resorbing ACS can lead to less than optimal hollow callus optimal architecture. In contrast, granulated calcium phosphate matrix or carriers with macroporosity in excess of 300 microns have been demonstrated to allow for rapid penetration of BMP induced blood vessels within the carrier leading to more uniform, mechanically superior, guided tissue repair callus constructs.

Optimal BMP carriers should also preferably be sufficiently cohesive and compression resistant to ensure a space for new bone formation without interference from surrounding soft tissues. This is particularly important for segmental defects and posterolateral spine fusion where soft tissues can protrude into the repair site.

### Example Constructs

The present invention encompasses a number of composite constructs that meet the design criteria discussed above. Table 1 sets forth several constructs according to various embodiments of the present invention. This listing is exemplary rather than comprehensive, and it will be appreciated that other constructs which meet the design criteria above are within the scope of the present invention.

TABLE 1
EXEMPLARY CONSTRUCTS

| | Design 1 | Design 2 | Design 3 | Design 4 | Design 5 |
|---|---|---|---|---|---|
| Biocompatible | rhCollagen | rhCollagen | rhCollagen | rhCollagen | rhCollagen |
| Matrix | sponge— | Sponge— | Sponge— | Sponge— | Sponge— |
| | 100-300 μm | 100-300 μm | 100-300 μm | 100-300 μm | 100-300 μm |
| | pore size | pore size | pore size | pore size | pore size |
| Granule Size | 425-800 μm, | 425-800 μm, | 425-800 μm, | 425-800 μm, | 425-800 μm, |
| & Geometry | angular | angular | angular | angular | angular |
| Granule Density | 0.200- | 0.200- | 0.200- | 0.200- | 0.200- |
| | 0.250 g/cc | 0.250 g/cc | 0.250 g/cc | 0.250 g/cc | 0.250 g/cc |
| Granule pH | 8.0-9.0 | 5.5-6.0 | 5.5-6.0 | 5.5-6.0 | 5.5-6.0 |
| Fenestrations | None | None | 1-2 mm | 1-2 mm | 1-2 mm |
| in Matrix | | | fenestrations | fenestrations | fenestrations |
| Surface Coating | None | None | None | Collagen | Collagen |
| Embedded | None | None | None | None | Poly- |
| Mesh | | | | | glycolide- |
| | | | | | co-lactide |
| | | | | | polymer |
| | | | | | mesh |

The biocompatible matrix of the constructs described in Table 1 is generally made from a composite slurry of rhCollagen and calcium phosphate granules. The collagen component is prepared by initiating fibrilogenesis of the pure rhCollagen material and inducing chemical crosslinking to generate a viscous solution. Porous calcium granules as described above are mixed with the collagen solution to form a slurry of defined concentration of both the collagen and ceramic constituents. This slurry can then be delivered into a mold of defined geometry to enable formation of the composite construct via additional chemical cross-linking and lyophilization steps. Tightly controlled lyophilization parameters result in the intrinsic 100-300 micron macroporous structure of the biocompatible matrix.

Some of the constructs set out in Table 1 include a second tier of macroporosity generated by including one or more fenestrations in the solid matrix. Generally, the fenestrations used in certain embodiments of the present invention are through-holes extending through at least one dimension of the construct. Without wishing to be bound by theory, the inventors believe that such fenestrations enhance tissue ingrowth and allow for a more uniformly distributed loading of osteoinductive material. Such fenestrations (if more than one) are preferably regularly spaced throughout the construct. Preferably, the size of each such fenestration is 1-2 millimeters in diameter. The inventors have found that a fenestration density of 5-20 fenestrations per square centimeter of overall matrix surface area is preferred, and more preferably, 7-10 fenestrations per square centimeter of overall matrix surface area. Alternatively, it is preferred that the matrix have 5-20% open area, and more preferably 12-16% open area, resulting from the fenestrations. Preferably, these fenestrations are made at the time of construct molding by using rods or pins around which the construct will form. After construct crosslinking and lyophilization, the rods or pins are removed from the mold leaving behind up to dozens to hundreds of open fenestrations in the construct which have the ability to retain their shape and maintain the macroporous structure. Alternatively, these fenestrations may be generated after the molding process is complete by punching or cutting the fenestrations using a die tool. Additionally, the fenestrations could be generated using high energy cutting technology such as laser or water jet cutting.

Some of the constructs detailed in Table 1 also include a surface coating applied to at least a portion of the outer surface thereof. Such coatings enable improved retention of the calcium phosphate granules within the matrix during handling and surgical implantation. The coating is, in some cases, similar or identical to the biocompatible matrix, and may be made from a similar or identical raw material as the biocompatible matrix, and may be similarly or identically cross-linked. Alternatively, the coating may differ in the degree of cross-linking (e.g. the coating may be non-cross-linked while the biocompatible matrix is cross-linked) or in some other manner (e.g. it may comprise a different collagen or a different polymer altogether).

Preferably the coating is applied to the constructs via a layering technique where the material for the coating is placed into a mold and the construct is layered down onto it. A second coating layer can then be applied to the top surface of the construct. The volume of the coating material is well controlled such that the average thickness of the surface coating is less than or equal to 0.5 mm. The coating can also be applied uniformly across a surface of the constructs with the aid of a spreader tool, or using a “painting” technique where the coating material is brushed/spread over the surface of the constructs. Further, spraying or spray-coating technologies may also be used to coat the constructs. Once the coating material has been applied to the construct, the entire assembly is lyophilized to its final form.

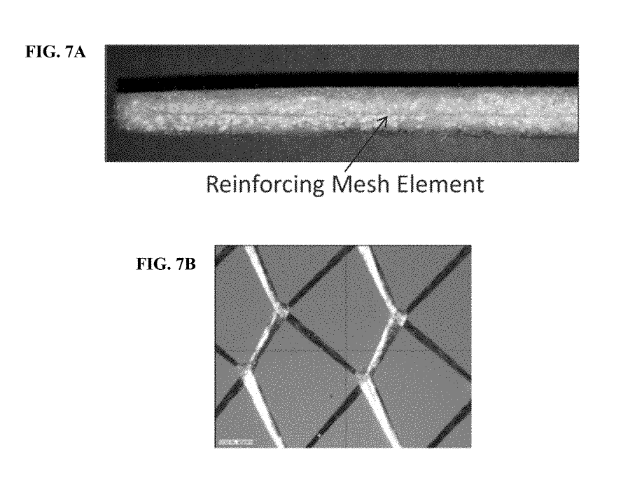

The tensile rigidity of the constructs is increased, in some embodiments, by the inclusion of one or more stiffening elements, such as one or more rods, fibers, or a mesh or braided element. Such stiffening elements are embedded within the composite constructs during the manufacturing process by placing them into the construct mold when applying the collagen/ceramic slurry. Preferably, the stiffening elements used in the present invention are mesh elements, such as that shown in FIG. 7B, having an outer dimension that substantially matches that of the construct into which they are incorporated. For example, a 100 mm (length)×25mm (width) construct would be embedded with a 100 mm (length)×25 mm (width) mesh element. The mesh elements of the present invention are preferably woven, braided or knitted (used interchangeably herein) from yarns of bioresorbable polymer fibers. Preferably, such mesh elements are characterized by openings having a maximum dimension of 1-2 mm. Preferably, the thickness of the stiffening elements used in the present invention is generally less than or equal to approximately 200 microns. The stiffening elements are, generally, formed from bioresorbable polymers such as those listed above, such as PGLA. The in vivo resorption rate of such polymers used in the stiffening elements of the present invention (including mesh elements) should be about the same as, or slower than, the resorption rate of the matrix into which they are placed. For example, such polymers (and therefore, such stiffening elements) should preferably take longer than thirty (30) days to fully resorb in vivo.

With or without the inclusion of such stiffening elements, constructs according to the various embodiments of the present invention are generally rigid enough to withstand the forces applied to them during and after implantation. Generally, constructs of certain embodiments of the present invention are characterized by a compression resistance (50%) of 30-250 kPa. The quantity of the ceramic granules embedded within the construct will contribute to the overall compressive properties of the construct. For example, a collagen sponge without any ceramic component will exhibit almost no compressive strength and can be readily deformed by soft tissues and fluid pressure near the anatomical site of implantation. As such, constructs according the embodiments of the present invention have been optimized such that quantity (density) of ceramic granules is tuned to provide sufficient compressive properties while still enabling the construct to retain the necessary macroporosity for promoting cell and tissue ingrowth. In certain embodiments of the present invention, the amount of granules in the construct is 150-310 milligrams per cubic centimeter (mg/cc) of matrix, preferably 200-250 mg/cc of matrix. These constructs, which are generally long, rectangular bodies, have been tested in a variety of animal models, including a rat muscle pouch model, primate fibula segmental defect and primate posterolateral spine fusion models; in all cases, constructs of the invention exhibit sufficient rigidity and resistance to deformation to remain in place without significant migration over intervals up to 26 weeks.

### EXAMPLE 1

### Scaffold Fabrication

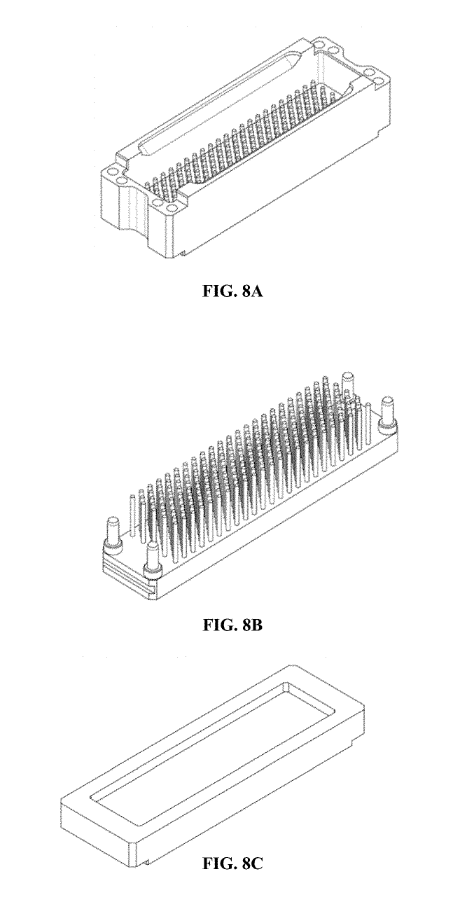

A collagen matrix material was prepared in accordance with the present invention. Collagen solution (3 mg/ml, 10 mM HCl) and 10× phosphate buffer (0.162 M Na₂HPO₄, pH 11.2) were mixed at a 9:1 (v:v) ratio, respectively, and stirred for one hour to obtain fibrillar collagen. The fibrillar collagen and a cross linking agent, 1-[3-(Dimethylamino) propyl]-3-ethylcarbodiimide (EDC) at a final concentration of 10 mM were mixed for two additional hours. The cross-linked collagen was collected by centrifugation (7300 g, 35 minutes) and washed by four cycles of re-suspension with purified water and centrifugation (7300 g, 35 minutes). The washed cross-linked collagen solution was finally adjusted to a concentration of 0.85% weight/weight. Calcium deficient hydroxyapatite (CDHA) granules (150 mg/ml) were added and mixed with the washed cross-linked collagen solution to obtain a core slurry. A first layer containing 7.2 g of the slurry was dispensed into a mold containing vertical protrusions or spikes (203 spikes, 1.5 mm diameter), as shown in FIG. 8A, per 100×25 mm mold. “Protrusions” and “spikes” are used synonymously herein to mean any elongated element that is used in the molding process of the present invention to create the fenestrations used in certain embodiments. A poly (glycolic-co-lactic acid) (PGLA) mesh, such as that shown in FIG. 7B, was placed onto the first slurry layer followed by second layer containing 7.2 g of the slurry dispensed on top of the mesh. An upper piece mold containing vertical spikes (matching the spikes in the bottom mold), as shown in FIG. 8B, was inserted on top of the completed core. The core sponge was then freeze-dried. Following freeze drying, the core sponge was subjected to a second step of cross linking by soaking in EDC solution (50 mM in 80% EtOH) for two hours. The cross-linked core sponge was extruded from the molds and extensively washed with purified water (seven exchanges).

A coating material for coating the cross-linked core sponge was prepared as follows. Collagen solution (3 mg/ml, 10 mM HCl) and 10× phosphate buffer (0.162 M Na₂HPO₄, pH 11.2) at a 9:1 ratio, respectively, were mixed and stirred for one hour to obtain fibrillar collagen. The fibrillar collagen was collected by centrifugation (7300 g, 35 minutes) and then was further washed by four cycles of re-suspension with purified water and centrifugation (7300 g, 35 minutes). The washed fibrillar collagen solution was finally adjusted to a concentration of 0.85% weight/weight. A first (bottom) coating layer containing 2 g of fibrillar collagen was dispensed on the bottom of a costume-made mold, as shown in FIG. 8C. The wet and washed cross-linked core sponge was placed on top of the bottom coating layer and a second coating layer containing 2 g of washed fibrillar collagen was dispensed on top of the core sponge. The coated sponge was then freeze-dried.

The PGLA bioresorbable polymer mesh incorporated into the core sponge was prepared as follows. PGLA yarn (28 filaments, 56 denier) having a diameter of about 0.175 millimeters was knitted into a mesh structure as shown in FIG. 7B. The resulting mesh was characterized as follows: thickness of 0.175 mm, aerial density of 16 g/m², pore size of 1.3 mm, tensile break of 30 lb, courses per inch (CPI) of 24.25, and wales per inch (WPI) of 14.75.

### EXAMPLE 2

### Efficacy in a Large Animal Spine Fusion Model

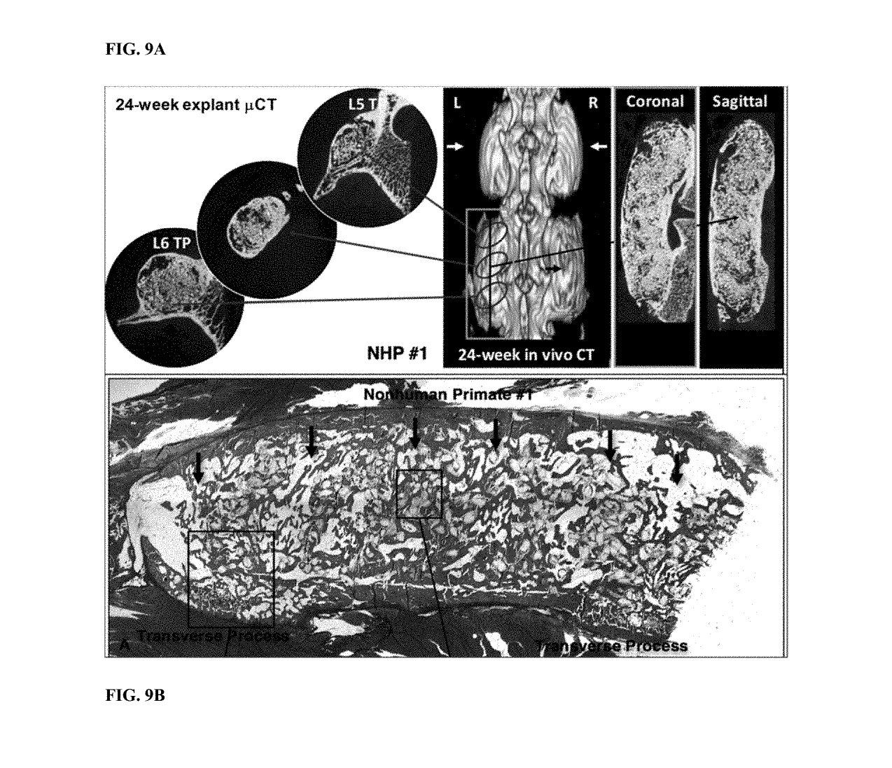

A composite collagen matrix was manufactured in accordance with the present invention as described in Design 5 (see Table 1). Samples of the collagen matrix were aseptically prepared for testing by trimming to 35 mm×10 mm×8 mm and applying an appropriate volume of liquid buffer containing an osteoinductive factor. The osteoinductive factor referred to as BMP-GER-NR in U.S. Pat. No. 8,952,131 was diluted in a pH 4 buffer to a concentration of 0.5 mg/mL. 3 mL of the protein/buffer solution per 10 cc of the composite collagen matrix was uniformly applied to the surface of the sample matrix and allowed to soak for at least 15 minutes. The properties of the calcium ceramic granules within the composite collagen matrix and this loading procedure enable the majority of the osteoinductive factor to become associated with the calcium ceramic granules. The final concentration of osteoinductive factor on the composite collagen matrix was 0.15 mg/cc. Samples were then implanted bilaterally in a non-human primate (rhesus macaque) model of uninstrumented posterolateral spine fusion. This animal model has been previously shown to be predictive of success in treating humans with a posterolateral spine fusion procedure. The transverse process of the L5-6 and/or L3-4 lumbar spine were exposed and decorticated with a mechanical burr. Implants were placed across adjacent transverse processes so as to span the spinal segment. The surgical site was closed according to standard veterinary surgical practice and animals were recovered and monitored for up to 24 weeks. The study was approved and conducted under IACUC guidelines. Radiographic and computed tomography (CT) imaging was performed throughout the study to monitor healing and the progress of spinal fusion. At the conclusion of the study the implants along with the associated spinal segments were retrieved and analyzed by micro-computed tomography (μCT) and histological sectioning. Increased radiodensity consistent with bone formation was observed within the implants over the course of the 24 week study. New bone formation could clearly be seen within the pore space of the 1-2 mm fenestrations described in this invention as well as throughout the interior of the composite collagen matrix. Bone was integrated with the adjacent bony tissue of the transverse processes and was continuous across the spinal segment, indicating a successful fusion. FIG. 9A shows an example CTs and 9B is an histological image from one animal illustrating the successful fusion achieved with the present invention.

### Conclusion

Throughout this application, reference is made to “macropores,” “micropores” and macro- and microporosity. In general, macropores have a cross-sectional dimension greater than 100 microns, while micropores are between 100 nm and 100 microns. Pores less than 100 nm are referred to as nanopores.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present invention have described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A biocompatible implant, comprising:
a matrix comprising cross-linked rhCollagen derived from a plant, said matrix having a density of 10-20 mg rhCollagen per cubic centimeter of the matrix and including a plurality of interconnected pores having a mean cross-sectional dimension between 100 and 300 microns;
a plurality of porous, calcium ceramic granules embedded within the matrix at a ratio of 150-310 milligrams of granules per cubic centimeter of matrix;
a bioresorbable polymer mesh within said matrix; and
a coating comprising rhCollagen and at least partially covering said matrix.

2. The implant of claim 1, wherein said matrix includes a plurality of fenestrations.

3. The implant of claim 2, wherein each fenestration of the plurality of fenestrations has a mean cross sectional dimension of 1-2 mm.

4. The implant of claim 3, wherein at least some of said plurality of fenestrations extend through a thickness of said matrix.

5. The implant of claim 1, further comprising a bone morphogenetic protein.

6. The implant of claim 5, wherein said bone morphogenetic protein is associated with said granules.

7. The implant of claim 1, wherein said granules have an average size within the range of 425 to 800 microns.

8. The implant of claim 1, wherein said mesh comprises a plurality of resorbable polymer fibers arranged in a yarn, and said yarn is fabricated into said mesh.

9. The implant of claim 8, wherein said mesh is characterized by openings with an average size of 1-2 millimeters.

10. The implant of claim 9, wherein said resorbable polymer comprises PGLA.

11. The implant of claim 1, wherein said calcium ceramic granules comprise calcium deficit hydroxyapatite.

12. A method of making a biocompatible implant, comprising the steps of:
forming a solution comprising cross-linked rhCollagen and a plurality of calcium ceramic granules;
transferring at least a portion of said solution within a mold to form a first slurry layer;
placing a bioresorbable polymer mesh onto said first slurry layer;
transferring at least a portion of said solution within the mold to form a second slurry layer and a preform comprising the first slurry layer, the bioresorbable polymer mesh, and the second slurry layer;
lyophilizing the preform;
contacting the preform with a coating material comprising rhCollagen, thereby at least partially coating the preform; and
lyophilizing the at least partially coated preform.

13. The method of claim 12, wherein the mold includes a plurality of protrusions.

14. The method of claim 12, further comprising the step of cross-linking the preform before said step of contacting the preform with a coating material.

15. The method of claim 14, wherein said step of cross-linking comprises soaking said preform with 1-[3-(Dimethylamino) propyl]-3-ethylcarbodiimide.

16. A kit comprising the implant of claim 1.

17. The kit of claim 16, further comprising a container of lyophilized osteoinductive protein.

18. A method of treating a patient, comprising the steps of:
contacting a bony tissue of the patient with a biocompatible implant, said implant comprising:
a matrix comprising cross-linked rhCollagen derived from a plant, said matrix having a density of 10-20 mg rhCollagen per cubic centimeter of matrix and including a plurality of interconnected pores having a mean cross-sectional dimension between 100 and 300 microns;
a plurality of porous, calcium ceramic granules embedded within the matrix at a ratio of 150-250 mg granules per cubic centimeter of matrix;
a bioresorbable polymer mesh within said matrix;
a coating comprising rhCollagen and at least partially covering said matrix; and
and an osteoinductive protein associated with the plurality of calcium ceramic granules.

19. The method of claim 18, wherein the protein-loaded biocompatible implant includes a plurality of fenestrations.

20. The method of claim 19, wherein each of the plurality of fenestrations has a mean cross sectional dimension of between 1-2 mm.

21. The implant of claim 18, wherein said granules have an average size within the range of 425 to 800 microns.

22. The implant of claim 18, wherein said mesh comprises poly-glycolide-co-lactide.

23. The implant of claim 18, wherein said calcium ceramic granules comprise calcium deficit hydroxyapatite.
